(19) United States
(12) Patent Application Publication
(10) Pub. No.: US 2004/0105892 A1
(43) Pub. Date: Jun. 3, 2004

(54) Use snake powder extract for treatment of cancer and HIV/AIDS

(76) Inventor: Pedro J. Lecca, Chevy Chase, MD (US)

Correspondence Address: Delphine James, Suite 170, 2656 South Loop West, Houston, TX, 77054, US

(21) Appl. No.: 10/306,958

(22) Filed: Dec. 2, 2002

(51) Int. Cl. A61K 35/58

(52) U.S. Cl. 424/542; 424/520

(57) ABSTRACT

A method of creating a composition of matter from a rattlesnake which is used as an immune system enhancer in human or animals. The method comprises providing the body of a rattlesnake which is continuously baked until the body of the rattlesnake completely dehydrates. Then, the dehydrated body is pulverized into a fine granular powder. The fine granular powder is then orally administered in an effective amount continuously and uninterrupted over a treatment period to a human or animal infected with cancer, AIDS or HIV.

### BACKGROUND

The present invention relates to the discovery of a chemical composition from the body of a rattlesnake which is an immune system enhancer. More particularly this invention relates to an animal product which is useful for the treatment and cure of cancer as well as HIV/AIDS treatment and cure. This invention also relates to a methodology for the treatment of carcinomas by administering the animal product in a designated manner. Clinical trials are being planned.

Cancer and HIV/AIDS are one of the major causes of death in the United States. Cancer is the new growth of tissue resulting from the continuous proliferation of abnormal cells that have the ability to invade and destroy other tissues and eventually cause death. Acquired Immune Deficiency Syndrome (AIDS) is a specific group of diseases or conditions that result from suppression of the immune system related to infection with the human immunodeficiency virus (HIV). With the loss of immune function, the person becomes susceptible to opportunistic infections (i.e. pneumonia, fungus infections, and other common ailments) that eventually cause death. When a person is HIV positive, it does not mean that he or she has AIDS. It could take more than ten years for a person to develop AIDS. Currently, the treatment of cancer is very invasive to the body and the treatment for AIDS only prolongs life with serious side effects.

The traditional methods of treating cancer are surgery, radiation, and chemotherapy. Surgery is the principle approach to curing cancer. However, for the success of surgery to cure cancer, the carcinomas must be caught early. If surgery cannot completely cure the cancer, surgery can be used in conjunction with radiation and chemotherapy. Ionizing radiation is utilized to destroy the cancerous growth. The abnormal cancerous tissue is more sensitive to the radiation than the normal tissue. Thus, radiation can be used to sterilize and to shrink the tumor, which then makes surgery possible. Chemotherapy is the use of drugs to treat cancer. The Chemotherapy drugs enter the blood stream and spreads throughout the body reaching cancerous growth that is inaccessible by surgery or radiation. The traditional methods show limited success and they destroy normal cells and can leave the patient handicapped.

Currently, there is no cure or vaccination effective against HIV. There are anti-viral drugs used to slow down the progression of AIDS and extends the life of AIDS patient.

The use of snakes to treat diseases has been utilized for over a decade. More particularly, there are several patents utilizing the snake venoms from various species of snakes. One example of the use of snake venom to treat cancer is described in U.S. Pat. No. 5,565,431. The snake venom is extracted to create an immune enhancer to help cure cancer through Immuno-therapy. Immuno-therapy, also called biologic therapy, uses the body's own immune system to fight cancer cells or protect the body from the side effects. Immuno-therapy relies on antibodies, which are naturally occurring proteins in the body dedicated to defending the body against invasion by foreign substances. In Immuno-therapy, the antibodies are used to attack the tumor cells directly. However, the use of the snake body to create an immune enhancer to help cure cancer and HIV/AIDS through Immuno-therapy is the novel discovery of this invention.

### SUMMARY

The present invention provides a method of creating a composition of matter from a rattlesnake that is an immune system enhancer in human or animals. The method comprise providing a body of a rattlesnake having a head, a midsection, and a tail, removing the head and the tail from the body, baking the midsection of the body at a temperature of at least 320 degrees until it is completely dehydrated, and pulverizing the dehydrated midsection of the body into a fine granular powder. The rattlesnake found to be most effective is from a species in the crotalus durissus family. The present invention provides a method of treating cancer, HIV, and AIDS by utilizing snake powder created by the above process. The method of the present comprises orally administering to an infected human an effective amount of the snake powder continuously and uninterrupted for a predetermined treatment period.

### BRIEF DESCRIPTION OF DRAWINGS

FIG. 1 illustrates the effect of Snake Extract on Tumor Cell Apoptosis using DMSO extract.

FIG. 2 illustrates the induction of cytotoxicity by snake extract using PBS solution.

FIG. 3 illustrates the induction of cytotoxicity by snake extract using DMSO solution.

FIG. 4 illustrates the induction of cytotoxicity by snake extract using PBS solution.

FIG. 5 illustrates the induction of cytotoxicity by snake extract using Ethanol solution.

FIG. 6 illustrates the effect of snake extract on tumor cell proliferation using DMSO extract.

FIG. 7 illustrates the effect of snake extract on tumor cell proliferation using Ethanol extract.

FIG. 8 illustrates the effect of snake extract on tumor cell proliferation using PBS extract.

Table 1 illustrates the results of utilizing the snake powder in three persons infected with terminal cancer.

### DETAILED SPECIFICATION

I have found evidence to show that the introduction of snake extract in a powdered form has produced some positive cellular activity associated with testing done with the extract in Ethanol, PBS, and DMSO solutions. Further testing has shown that when this powdered form was administered in controlled amounts to three people with various forms of terminal cancer, marked improvement was shown over the span of three months to one and one-half years.

The present invention is based on the applicant's research of the practices by Latin American tribes utilizing snake methodology for healing various illnesses. It is my contention that capturing the rattlesnake in question and reducing it to a powder component, will allow it to be used for cancer and HIV treatment. The key to this concept is in using the entire body of the animal, not just the venom, which has been used in prior studies.

The process of the present invention first requires that the captured rattlesnake's (Cascabel-Tzabean) head and tail be removed from the rest of the body. The remaining mid-section of the body of the rattlesnake is then baked in an oven at a temperature of at least 320 degrees. The remaining mid-section is baked until it is dehydrated. After the mid-section is fully dehydrated, it is then pulverized into a powder form. The Crotalus Durissus species of the rattlesnake has shown to be the most effective against the treatment of cancer.

Initially, the powdered form was tested in Ethanol, PBS, and DMSO solutions as described below:

1. Induction of cytotoxicity by snake extract in Ethanol solution, PBS solution and DMSO solution

2. Effect of snake extract on tumor cell proliferation in Ethanol solution, PBS and DMSO solution

3. Effect of snake extract on tumor cell apoptosis in DMSO solution

The following cell lines were utilized in the tests listed above: Leukemia/Lymphoma: K562, Raji, Jurkatt, CEM; Breast Carcinoma MCF-7, MDA-MB-231, MD-MB-468; Prostate Carcinoma LNCaP, PC-3; Colorectal Carcinoma: HT-29, HCT-116; Pancreatic Carcinoma: PANC-1; Gliobastaoma: GL10-1.

Laboratory analysis utilizing the PBS solution, ethanol solution, DMSO solution on the above listed cell lines has been found to interfere with the effect of the positive benefits of the powder for the treatment of cancer. However, even with the diminished effects brought on by the use of solution mediums, the positive effects of the snake powder was not totally destroyed.

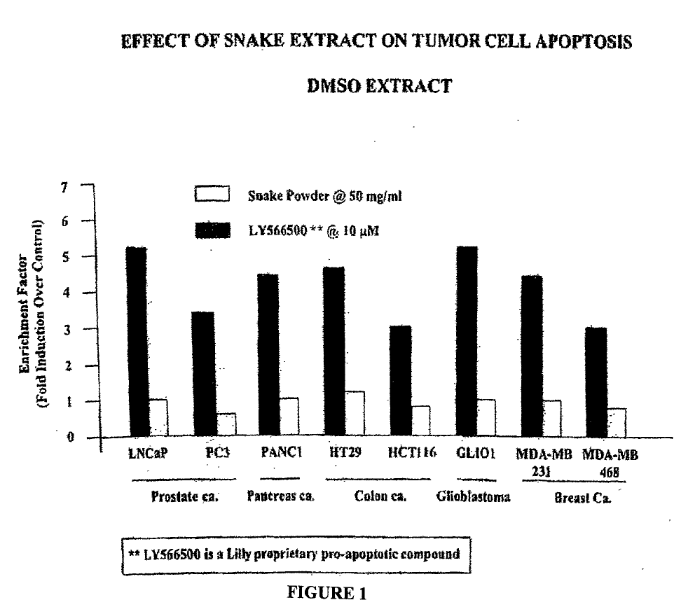

In reference to FIG. 1, the effect of the snake extract on tumor cell apoptosis with dimethysulfoxide (DMSO) solution is analyzed. Apoptosis has been defined as a form of programmed cell death and it plays a major role during development as a major mechanism for the precise regulation of cell numbers and as a defense mechanism to remove unwanted and potentially dangerous cells that have been infected by viruses and tumor cells. In all the cancer cell lines investigated, as shown in FIG. 1, LY566500 (Lilly proprietary pro-apoptotic compound) appeared to be better able to cause cell death (apoptosis) than the snake powder. However, the snake powder did show some marked activity of cell death (apoptosis) in each cell line investigated.

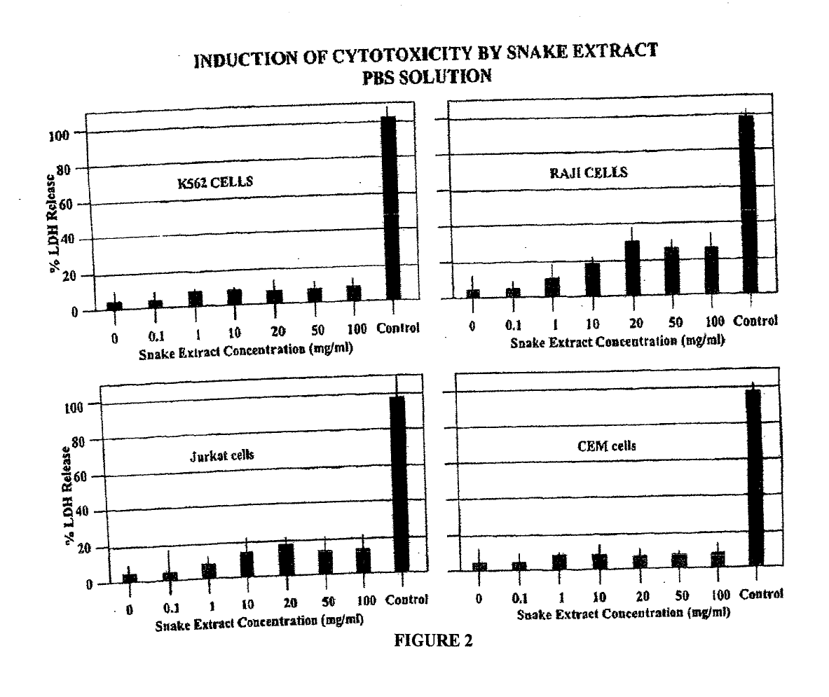

In reference to FIGS. 2, 3, and 4 the induction of cytotoxicity by snake extract utilizing the PBS solution, DMSO, and ethanol solutions are respectively analyzed. It is known that small amounts of enzymes are present in the blood circulation at all times. Lactate dehydrogenase (LDH) is one of these enzymes. LDH catalyzes the conversion of lactate and pyruvate. Consequently, LDH represents a group of enzymes that are present in almost all metabolizing cells and about five individual isoenzymes make up the total LDH serum level. When tissue damage occurs, the LDH is released into the blood in larger quantities. In fact, the serum levels of the enzymes are often used as an aid in the diagnosis of certain diseases Thus, the release of LDH is analyzed

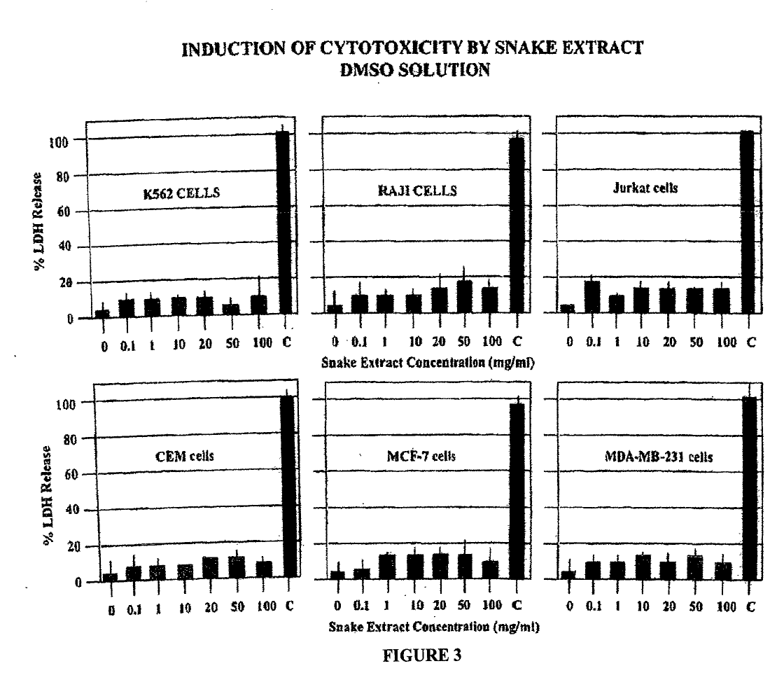

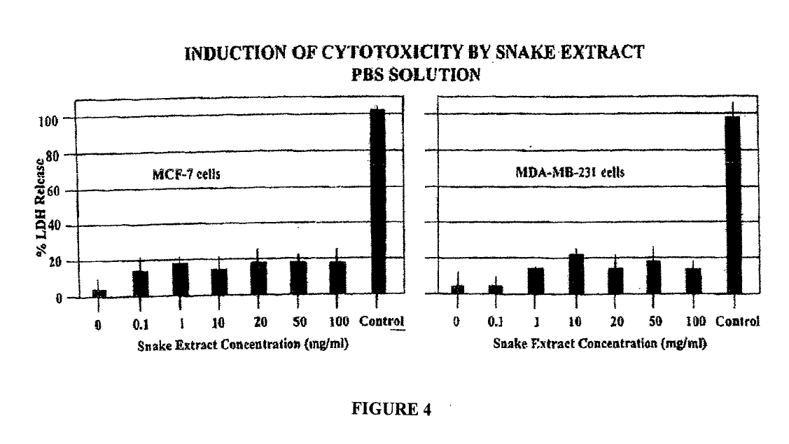

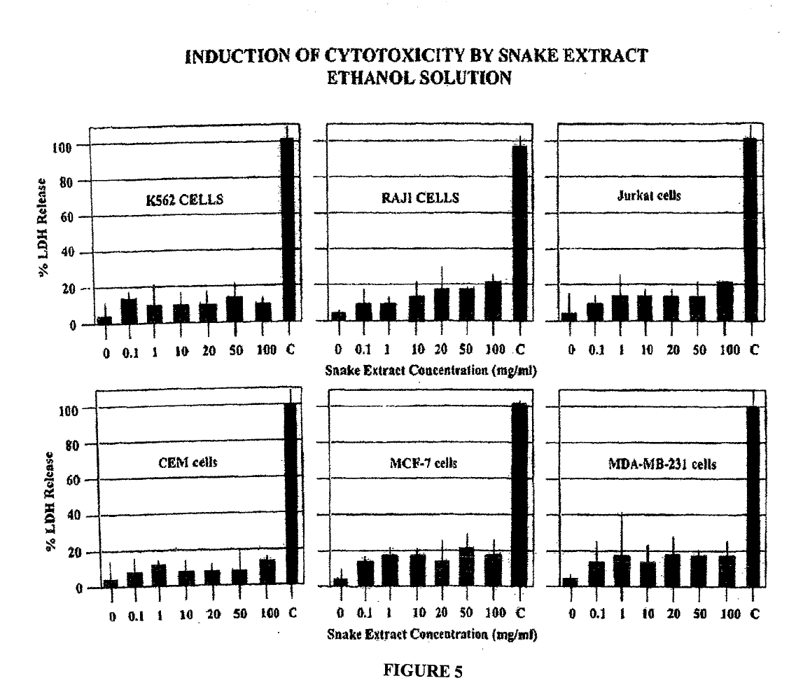

Referring to FIG. 2, the snake extract prepared using PBS buffer, at all concentrations investigated, was less injurious to different types of cell lines because it caused less percent release of LDH. As shown in FIG. 3, a similar pattern was observed when cytotoxicity was induced utilizing the snake extract in the DMSO solution. As shown in FIG. 4, a similar pattern was observed when cytotoxicity was induced utilizing the snake extract in the PBS solution against additional cell lines. As shown in FIG. 5, a similar pattern was observed when cytotoxicity was induced utilizing the snake extract in the ethanol solution.

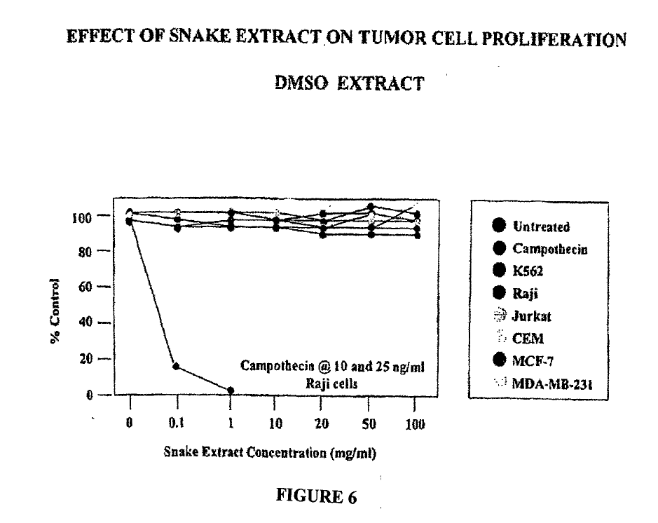

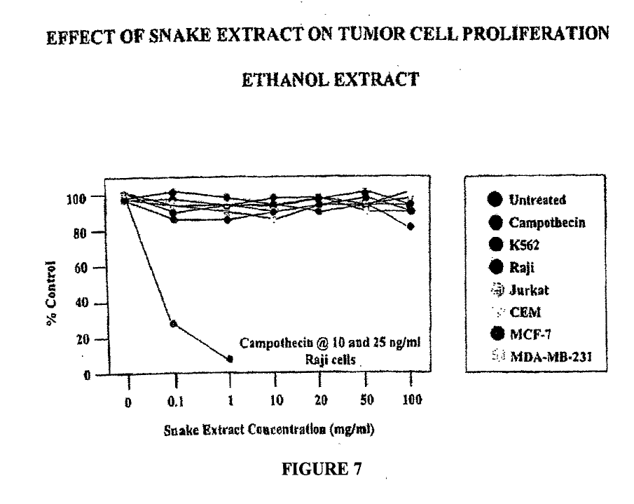

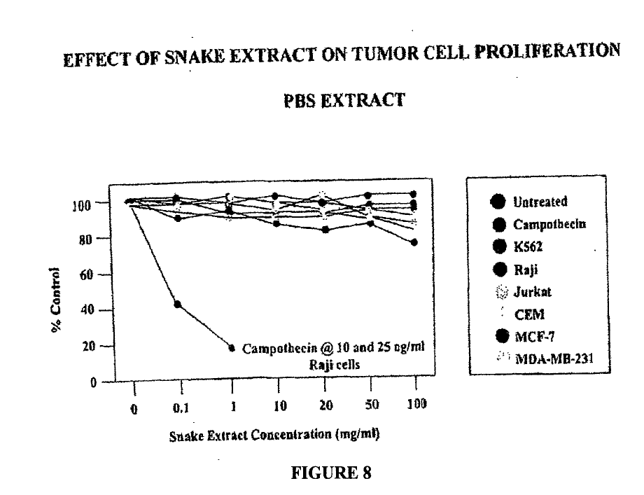

Referring to FIGS. 6, 7, and 8 the effect of snake extract on tumor cell proliferation utilizing the DMSO, ethanol, and PBS solutions, is respectively analyzed. Camptothecin is known to be active against tumors that are normally considered chemorefractory such as colorectal and lung tumors. Camptothecin inhibits topoisomerase I, an enzyme critical to the growth of tumor cells. Further, Camptothecin can also cause single strand breaks in DNA. Referring to FIGS. 6, 7, and 8, Camptothecin is more effective at inhibiting cell proliferation of different cell lines than the snake powder extract. However, in each case analyzed the snake extract showed some marked activity when compared with untreated cell lines. In each case analyzed in FIGS. 1-8, the snake powder extract produced some positive cellular activity associated with testing done in the ethanol, PBS, and DMSO solutions. It was concluded that the solvents might interfere with activity of the snake powder extract; thereby causing reduced activity.

Further experiments were done utilizing laboratory rats to analyze the anti-free radical and immune function properties of the pure snake powder. Pure snake powder was injected into the blood stream of laboratory rats. Results showed that the activity of sodium dismutase enzyme (SOD) in the erythrocytes of rats increased. The T-lymphocyte transformation in the peripheral blood of the rats increased. It was concluded that the pure snake powder has anti-free radical action and can increase immune function in experimental animals.

Further testing using the undiluted powder by three persons having various forms of terminal cancer was conducted. The results of these tests showed a significant increase in the ability of each person's immune system to naturally inhibit the growth of cancer tumor cells. The results of the tests are shown in Table 1.

Referring to the first cancer patient in Table 1, this patient took the snake powder after completing chemotherapy and radiation therapy. The patient is Male, was born on Jul. 25, 1942, and had head and neck cancer. Patient 1 was diagnosed with head and neck cancer and had received chemotherapy, (drugs) and radiation treatments with no positive results. When the first patient started taking the snake powder on Jul. 21, 2001, he could not swallow. On Jul. 28, 2001, swallowing improved. After taking the snake powder for six weeks with no other medication or treatment, his condition improved. A CT-Scan of the neck was performed which showed no lymphadenopathy, no abnormal mass, or no enhancement within the neck area Additionally, Patient 1 had blood cultures that indicated the cancer was not spreading and was reduced. By Sep. 3, 2001, this Patient swallowing was normal.

In reference to the second cancer patient in Table 1, the patient was a 28 year old female who was diagnosed in March of 2000 through blood tests that indicated a positive test for Leukemia. Blood transfusions were given once a week for 14 months. Additionally, during the months of April 2000 and May 2000, the patient was treated with dialysis. The Leukemia remained with no satisfactory remission at that time.

The second patient began taking the snake powder in March 2001 and continued until June 2001. After her treatment period, a blood test was taken which showed remission of her cancer. Another blood test was performed in August 2002, which again showed no cancer cells in the blood. In reference to the third cancer patient, the patient was a 34 year old male with prostate cancer identified in 1997. He was treated with chemotherapy for two years with no positive result, with continued weight loss and pain. The patient started taking the snake powder in late October 1999 until December 1999. After the treatment period, his blood test was normal, and the cancer tumor in the prostate area was eradicated. He was able to return to work.

From the above experiments, it is my contention that the ingredients in the snake powder enhance the immune system to prevent the cancer cells from growing and allowing the natural mechanisms of body system (i.e. the white blood cell), to attack and to kill the cancer cells. Since AIDS/HIV attacks the immune system, it can be concluded that the snake powder can be used to enhance the immune system to treat AIDS/HIV.

TABLE 1

| | | | | | Snake Powder Treatment | |
|---|---|---|---|---|---|---|
| | | | | Traditonal | Period | |
| Patient | Age | Gender | Type of cancer/Diagnosis | Treatment Period | Time Elapsed | Results |
| 1 | 56 | Male | TXN 3 MO Squamous Cell Carcinoma of the | Radiation and | July 2001 to | Cat-scan showed no |
| | | | head and neck | chemo-therapy | September 2001 | tumor and |
| | | | Tumor at base of tongue and right and left side | March 1998 to May 1998 | Treatment amount - | 100% remission |
| | | | of neck. | Recurrence in March 1999 | ½ tsp | |
| | | | Could not swallow. | | once a day | |
| | | | | Blood transfusion given | | |
| 2 | 28 | Female | Leukemia | once a week | March 2000 to | Blood tests taken |
| | | | Blood tests indicated a positive test for | Dialysis | June 2001 | showed remission |
| | | | Leukemia | April 1999 to | Treatment amount - | until there were no |
| | | | | May 2000 | ½ tsp | cancer cells |
| | | | | No remission | once a day | observed in |
| 3 | 34 | Male | Prostate Cancer | Chemotherapy | October 1999 to | Blood tests were |
| | | | Identified in 1997. Patient was losing weight | Two years | December 1999 | normal. Cancer |
| | | | and had some | No positive result. | Treatment amount - | tumor in prostate |
| | | | discomfort. | Continued weight loss and | ½ tsp | was gone. |
| | | | | pain | once a day | |

What is claimed is:

1. A method of creating a composition of matter from a rattlesnake that is an immune system enhancer in human or animals, the method comprising:
providing a body of a rattlesnake having a head, a midsection, and a tail;
removing the head and the tail from the body;
baking the midsection of the body until it is completely dehydrated; and
pulverizing the dehydrated midsection of the body into a fine granular powder.

2. The method of claim 1 wherein the rattlesnake is a species in the crotalus durissus family.

3. The method of claim 1 wherein the step of baking is carried on at a temperature of at least 320 degrees.

4. The product produced by the process of claim 2.

5. A method of treatment or inhibiting cancer in a human or animal which comprises orally administering an effective amount of the product of claim 4 continuously and uninterrupted for a predetermined treatment period.

6. A method of treatment or inhibiting AIDS in a human or animal which comprises orally administering an effective amount of the product of claim 4 continuously and uninterrupted for a predetermined treatment period.

7. A method of treatment or inhibiting HIV in a human or animal which comprises orally administering an effective amount of the product of claim 4 continuously and uninterrupted for a predetermined treatment period.
